(19) United States
(12) Patent Application Publication
(10) Pub. No.: US 2024/0024027 A1
(43) Pub. Date: Jan. 25, 2024

(54) MODULATING SURGICAL DEVICE SETTINGS BASED ON FORECASTED SURGICAL SITE CONDITIONS

(71) Applicant: GYRUS ACMI, INC. D/B/A OLYMPUS SURGICAL TECHNOLOGIES AMERICA, Westborough, MA (US)

(72) Inventors: Kurt G. Shelton, Bedford, MA (US); Kristen R. Toutant, Denver, CO (US)

(21) Appl. No.: 18/350,912

(22) Filed: Jul. 12, 2023

Related U.S. Application Data
(60) Provisional application No. 63/369,098, filed on Jul. 22, 2022.

(51) Int. Cl. A61B 18/26

(52) U.S. Cl. CPC A61B 18/26; A61B 2018/00791

(57) ABSTRACT

Systems and methods for automatic control of surgical site temperature during an endoscopic procedure are disclosed. An exemplary endoscopic surgical system comprises a endoscopic surgical device controllably coupled to a medical instrument to deliver energy to an anatomical target at a surgical site, a temperature sensor to measure temperatures at the surgical site at different times during the procedure, and a controller circuit to generate a temperature trend or a prediction of future temperature at the surgical site using the temperature measurements. Based at least in part on the generated temperature trend or the prediction of future temperature at the surgical site, the controller circuit can adjust at least one operating parameter associated with the endoscopic surgical system to achieve or maintain substantially a desired temperature at the surgical site during the procedure to prevent, or reduce the severity of, laser-induced tissue thermal damage.

### PRIORITY CLAIM

This application claims the benefit of priority to U.S. Provisional Patent Application Ser. No. 63/369,098, filed Jul. 22, 2022, the contents of which are incorporated herein by reference.

### TECHNICAL FIELD

This invention relates generally to an endoscopic surgical system, and more specifically relates to approaches for modulating one or more settings of the endoscopic surgical system based on a forecasted surgical site condition.

### BACKGROUND

Endoscopes are typically used to provide access to an internal location of a patient so that a doctor is provided with visual access. Some endoscopes are used in minimally invasive surgery to remove unwanted tissue or foreign objects from the body of the patient. For example, a nephroscope is used by a clinician to inspect the renal system, and to perform various procedures under direct visual control. In a percutaneous nephrolithotomy (PCNL) procedure, a nephroscope is placed through the patient's flank into the renal pelvis. Calculi or mass from various regions of a body including, for example, urinary system, gallbladder, nasal passages, gastrointestinal tract, stomach, or tonsils, can be visualized and extracted.

Various medical instruments such as laser or plasma systems have been used for delivering surgical laser energy to various target treatment areas such as soft or hard tissue. Examples of the laser treatment include ablation, coagulation, vaporization, fragmentation, etc. In lithotripsy applications, laser has been used to break down calculi structures in kidney, gallbladder, ureter, among other stone-forming regions, or to ablate large calculi into smaller fragments. The calculi fragments may be removed via a working channel of an endoscope (e.g., an ureteroscope) or may be passed naturally by the patient following the procedure.

Heat buildup is a potentially hazardous consequence of laser irradiation of an anatomical or calculi target, particularly in cases where relatively high intensity laser output is used in the treatment, such as laser lithotripsy to ablate or fragment a calculi target of certain size, shape, hardness, or composition. Excessive heat buildup at or near the surgical site may cause thermal damage of non-targeted tissue or organs.

### SUMMARY

Effective surgical site temperature control can help prevent tissue thermal damage caused by heat buildup during medical procedures such as laser lithotripsy or ultrasound lithotripsy procedures. Conventionally, temperature is sensed from a surgical site and displayed to a user (e.g., a physician) during the procedure. The user can manually change settings of the medical instruments (e.g., laser output intensity), or temporarily turning off the medical instruments, if the surgical site temperature reaches or exceeds a safety limit. Such manual temperature adjustment may not provide precise temperature control at the surgical site. Additionally, adjustment of the settings of the medical instruments (e.g., laser output intensity) may not achieve adequate and fast temperature relief at the surgical site. For example, in some cases, reducing laser output intensity or shutting off laser output may compromise therapy efficiency and/or extend procedure time. It should be noted that the medical instrument herein refers to a laser system, but any suitable medical instruments, such as an ultrasound system, which may be coupled to or implemented in an endoscope for providing treatment or diagnosis of a target are also within the scope of the present invention.

The present invention describes systems, devices, and methods to improve surgical site temperature control by automatically adjusting one or more device settings based on forecasted surgical site conditions, such as a temperature trend or a prediction of future temperature at or near the surgical site. According to one embodiment, an exemplary endoscopic surgical system comprises a endoscopic surgical device controllably coupled to a medical instrument (e.g., a laser system) and configured to deliver energy (e.g., laser energy) to an anatomical target at a surgical site during a procedure, a temperature sensor configured to measure temperatures of the surgical site at different times during the procedure; and a controller circuit to generate a temperature trend or a prediction of future temperature in a vicinity of the surgical site using the temperature measurements at the different times. Based at least in part on the generated temperature trend or the prediction of future temperature at the surgical site, the controller circuit can adjust at least one operating parameter associated with the endoscopic surgical system to achieve or maintain substantially a desired temperature at the surgical site during the procedure. In this document, the term “substantially” means±10%, and in some embodiments, ±5%. The surgical site temperature control approach described herein may advantageously prevent, or reduce the severity of, tissue thermal damage caused by excessive energy (e.g., laser energy) delivered to the tissue site. Various temperature control means allow for more versatile control of surgical site temperature in accordance with the surgical site conditions. The alternative temperature control means (e.g., irrigation or suction flow and irrigant treatment) can help avoid discontinuation or substantial reduction of energy output in an endoscopic procedure (e.g., a laser or ultrasound lithotripsy procedure). As such, more precise and faster temperature control and improved laser therapy efficacy and tissue safety can be achieved.

Example 1 is an endoscopic surgical system, comprising: an endoscopic surgical device controllably coupled to a medical instrument for delivering energy to an anatomical target at a surgical site during a procedure; a temperature sensor for measuring temperatures in a vicinity of the surgical site at different times during the procedure; and a controller circuit configured to: generate a temperature trend or a prediction of future temperature at the surgical site based at least in part on the temperature measurements at the different times; and based at least in part on the generated temperature trend or the prediction of future temperature at the surgical site, adjust at least one operating parameter associated with the endoscopic surgical system to achieve or maintain substantially a desired temperature at the surgical site during the procedure.

In Example 2, the subject matter of Example 1 optionally includes, wherein the medical instrument comprises at least one laser system for delivering laser energy to a calculi target at the surgical site when the endoscopic surgical system operates in accordance with the adjusted at least one operating parameter.

In Example 3, the subject matter of any one or more of Examples 1-2 optionally includes, wherein the controller circuit is further configured to: determine a temperature change rate at the surgical site using the generated temperature trend; and adjust the at least one operating parameter in response to the determined temperature change rate exceeding a predetermined threshold of a temperature change rate.

In Example 4, the subject matter of any one or more of Examples 1-3 optionally includes, wherein the controller circuit is further configured to: generate a trained prediction model using the temperature measurements at the different times; generate the prediction of future temperature at the surgical site further using the trained prediction model; and adjust the at least one operating parameter in response to the prediction of future temperature exceeding a temperature threshold.

In Example 5, the subject matter of any one or more of Examples 1-4 optionally includes, wherein the controller circuit is further configured to: estimate a safe-operation time window using the temperature measurements at the different times, the safe-operation time window representing an estimate of time taken to reach a safe-operating temperature limit at the surgical site; and adjust the at least one operating parameter in response to the estimated safe-operation time window falling below a time threshold.

In Example 6, the subject matter of Example 2 optionally includes, wherein the at least one operating parameter to be adjusted includes a laser output setting of the at least one laser system.

In Example 7, the subject matter of Example 6 optionally includes, wherein to adjust the laser output setting, the controller circuit is further configured to toggle between at least first and second pre-determined pulse profiles based at least in part on the generated temperature trend or the prediction of future temperature at the surgical site, the second pre-determined pulse profile having a lower average power than the first pre-determined pulse profile.

In Example 8, the subject matter of any one or more of Examples 6-7 optionally includes, wherein to adjust the laser output setting, the controller circuit is further configured to reduce an average power of laser pulses delivered to the surgical site in response to (i) the generated temperature trend indicating an increase in temperature at a rate exceeding a rate threshold, or (ii) the prediction of future temperature exceeding a temperature threshold.

In Example 9, the subject matter of Example 8 optionally includes, wherein to reduce the average power of laser pulses includes to reduce at least one of: a pulse width of a laser pulse; a peak power of a laser pulse; and a pulse frequency representing a number of laser pulses per unit time.

In Example 10, the subject matter of any one or more of Examples 1-9 optionally include an irrigation and/or suction system configured to provide irrigant into, and suction of fluid from, the surgical site.

In Example 11, the subject matter of Example 10 optionally includes, wherein to adjust the at least one operating parameter, the controller circuit is further configured to adjust, via the irrigation and/or suction system, at least one of an irrigation flow or a suction flow based at least in part on the generated temperature trend or the prediction of future temperature at the surgical site.

In Example 12, the subject matter of Example 11 optionally includes, wherein the controller circuit is configured to increase at least one of the irrigation flow or the suction flow in response to (i) the generated temperature trend indicating an increase in temperature at a rate exceeding a rate threshold, or (ii) the prediction of future temperature exceeding a temperature threshold.

In Example 13, the subject matter of any one or more of Examples 11-12 optionally includes a pressure sensor configured to sense a pressure at the surgical site during the procedure, wherein the controller circuit is further configured to selectively increase the irrigation flow or the suction flow via the irrigation and/or suction system, including to: increase the suction flow or decrease the irrigation flow when the sensed pressure exceeds an upper pressure limit; increase one or both of the irrigation flow or the suction flow when the sensed pressure is within a range defined by the upper pressure limit and a lower pressure limit; and increase the irrigation flow or decrease the suction flow when the sensed pressure falls below the lower pressure limit.

In Example 14, the subject matter of any one or more of Examples 10-13 optionally include an irrigant treatment unit configured to alter a temperature of the irrigant, wherein the controller circuit is further configured to generate a control signal to the irrigant treatment unit to adjust a temperature of the irrigant before reaching the surgical site based at least in part on the generated temperature trend or the prediction of future temperature at the surgical site.

In Example 15, the subject matter of Example 14 optionally includes, wherein the irrigant treatment unit includes a cooling system configured to, under the control of the controller circuit, cool the irrigant before reaching the surgical site in response to (i) the generated temperature trend indicating an increase in temperature at a rate exceeding a rate threshold, or (ii) the predicted future temperature exceeding a temperature threshold.

In Example 16, the subject matter of any one or more of Examples 14-15 optionally include, wherein the irrigant treatment unit includes a fluid mixer configured to, under the control of the controller circuit, mix at least two irrigant sources of different temperatures before reaching the surgical site in response to (i) the generated temperature trend indicating an increase in temperature at a rate exceeding a rate threshold, or (ii) the predicted future temperature exceeding a temperature threshold.

In Example 17, the subject matter of any one or more of Examples 2 and 6-9 optionally include, wherein the endoscopic surgical device includes an optical pathway with an adjustable distal portion, the optical pathway configured to direct the laser energy to the anatomical target, wherein the controller circuit is further configured to, based at least in part on the generated temperature trend or the prediction of future temperature at the surgical site, generate a control signal to an actuator coupled to the optical pathway to adjust a position or orientation of the distal portion of the optical pathway relative to the anatomical target.

In Example 18, the subject matter of any one or more of Examples 1-17 optionally includes, wherein the at least one operating parameter associated with the endoscopic surgical system comprises at least one of: a temperature of an irrigant before being applied to the surgical site; an irrigation flow rate; a suction flow rate; or a laser output setting of a laser system.

In Example 19, the subject matter of Example 18 optionally includes, wherein the controller circuit is further configured to perform the adjustment with a bias toward one of the operating parameters based at least in part on at least one of the generated temperature trend, the prediction of future temperature, or a pressure at the surgical site.

In Example 20, the subject matter of Example 19 optionally includes, wherein the controller circuit is further configured to, upon determining that the pressure at the surgical site is substantially below a maximal allowable pressure, adjust at least one of the irrigation flow rate or the suction flow rate prior to adjusting the laser output setting.

In Example 21, the subject matter of any one or more of Examples 19-20 optionally includes, wherein the controller circuit is further configured to, upon determining that the pressure at the surgical site is substantially close to a maximal allowable pressure, adjust the laser output setting prior to adjusting the irrigation flow rate or the suction flow rate.

In Example 22, the subject matter of any one or more of Examples 1-21 optionally include a user interface device configured to generate an alert of surgical site temperature rise in response to (i) the generated temperature trend indicating an increase in temperature at a rate exceeding a rate threshold, or (ii) the prediction of future temperature exceeding a temperature threshold.

In Example 23, the subject matter of Example 22 optionally includes, wherein the user interface device is configured to generate a recommended adjustment of the at least one operating parameter, and to receive a user input to confirm, reject, or modify the recommended adjustment.

Example 24 is a method for controlling temperature at a surgical site of a patient during an endoscopic procedure using an endoscopic surgical system, the method comprising: directing energy produced by a medical instrument to an anatomical target at the surgical site; measuring temperatures in a vicinity of the surgical site at different times during the procedure; generating a temperature trend or a prediction of future temperature at the surgical site based at least in part on temperature measurements at the different times; and based at least in part on the generated temperature trend or the prediction of future temperature at the surgical site, adjusting at least one operating parameter associated with the endoscopic surgical system to achieve or maintain substantially a desired temperature at the surgical site during the procedure.

In Example 25, the subject matter of Example 24 optionally includes determining a temperature change rate at the surgical site using the generated temperature trend, wherein adjusting the at least one operating parameter is in response to the determined temperature change rate exceeding a predetermined threshold of a temperature change rate.

In Example 26, the subject matter of any one or more of Examples 24-25 optionally include generating a trained prediction model using the temperature measurements at the different times, wherein generating the prediction of future temperature at the surgical site is by using the trained prediction model, wherein adjusting the at least one operating parameter is in response to the prediction of future temperature exceeding a predetermined temperature threshold.

In Example 27, the subject matter of any one or more of Examples 24-26 optionally include estimating a safe-operation time window using the temperature measurements at the different times, the safe-operation time window representing an estimate of time taken to reach a safe-operating temperature limit at the surgical site, wherein adjusting the operating parameter is in response to the estimated safe-operation time window falling below a time threshold.

In Example 28, the subject matter of any one or more of Examples 24-27 optionally includes, wherein adjusting the at least one operating parameter includes, via at least one laser system, reducing an average power of laser pulses delivered to the surgical site in response to (i) the generated temperature trend indicating an increase in temperature at a rate exceeding a rate threshold, or (ii) the prediction of future temperature exceeding a temperature threshold.

In Example 29, the subject matter of any one or more of Examples 24-28 optionally includes, wherein adjusting the at least one operating parameter includes, via an irrigation and/or suction system, increasing at least one of an irrigation flow of irrigant into the surgical site or a suction flow of fluid out of the surgical site, in response to (i) the generated temperature trend indicating an increase in temperature at a rate exceeding a rate threshold, or (ii) the prediction of future temperature exceeding a temperature threshold.

In Example 30, the subject matter of Example 29 optionally includes sensing a pressure at the surgical site during the procedure using a pressure sensor, wherein adjusting at least one of the irrigation flow or the suction flow includes: increasing the suction flow or decrease the irrigation flow when the sensed pressure exceeds an upper pressure limit; increasing one or both of the irrigation flow or the suction flow when the sensed pressure is within a range defined by the upper pressure limit and a lower pressure limit; and increasing the irrigation flow or decrease the suction flow when the sensed pressure falls below the lower pressure limit.

In Example 31, the subject matter of any one or more of Examples 24-30 optionally includes, wherein adjusting the at least one operating parameter includes adjusting, via an irrigant treatment unit coupled to an irrigation and/or suction system, a temperature of an irrigant before flowing into the surgical site based at least in part on the generated temperature trend or the prediction of future temperature at the surgical site.

In Example 32, the subject matter of any one or more of Examples 24-31 optionally includes, wherein adjusting the at least one operating parameter includes adjusting a position or orientation of a distal portion of an optical pathway relative to the anatomical target at the surgical site, and directing the energy to the anatomical target via the optical pathway.

In Example 33, the subject matter of any one or more of Examples 24-32 optionally includes, wherein the at least one operating parameter associated with the endoscopic surgical system comprises at least one of: a temperature of an irrigant before being applied to the surgical site; an irrigation flow rate; a suction flow rate: or a laser output setting of a laser system.

In Example 34, the subject matter of any one or more of Examples 24-33 optionally includes, on a user interface: generating an alert of surgical site temperature rise in response to (i) the generated temperature trend indicating an increase in temperature at a rate exceeding a rate threshold, or (ii) the prediction of future temperature exceeding a temperature threshold; and generating a recommended adjustment of the at least one operating parameter and receiving a user input to confirm, reject, or modify the recommended adjustment.

This summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 is a block diagram illustrating an example of a laser energy delivery system configured to provide laser treatment to an anatomical target at or near a surgical site.

FIG. 2 is a block diagram illustrating an endoscopic surgical system with automatic surgical site condition control, and at least a portion of the environment in which the system may operate.

FIG. 3 illustrates an example of an endoscopic laser lithotripsy system with automatic surgical site condition control.

FIG. 4 illustrates an example of a temperature trend and a prediction of a future temperature using the trend.

FIG. 5 is a flowchart illustrating an example method of controlling surgical site temperature during an endoscopic procedure for treating an anatomical target.

FIG. 6A is a flowchart illustrating an example method of generating a temperature management plan based on surgical site conditions such as surgical site temperature and surgical site pressure.

FIG. 6B is a flowchart illustrating an example of generating a temperature management plan with prioritized means for controlling surgical site temperature.

FIG. 7 is a block diagram illustrating an example machine upon which any one or more of the techniques (e.g., methodologies) discussed herein may perform.

### DETAILED DESCRIPTION

An endoscopic procedure is a medical procedure of viewing and operating on an internal organ, and/or delivering energy (e.g., laser energy or ultrasound energy) to a target body region to achieve a particular diagnostic or therapeutic effect. For example, laser endoscopy have been used for treatment of soft and hard tissue (e.g., damaging or destroying cancer cells), or in lithotripsy applications. During the procedure, a practitioner can insert a scope through an incision in a patient's ureter and into the patient's kidney. Through the scope, the practitioner can locate certain stones in the kidney or upper ureter, break the stones into smaller fragments by illuminating the stone, through the scope, with relatively high-powered infrared laser beam. The laser beam can ablate a stone into smaller fragments. The stone fragments can then be withdrawn from the kidney. The scope can include an endoscope, a nephroscope, and/or a cystoscope.

Laser energy delivered to the environment of the surgical site and laser treatment of anatomical target (e.g., ablation and fragmentation of a calculi target) may cause heat buildup at or near the surgical site, particularly in cases where relatively high intensity laser output is used, such as to ablate or fragment a calculi target of certain size, hardness, or composition. To prevent hazardous consequences such as tissue thermal damage, intracorporeal or surgical-site temperature can be monitored during the procedure to ensure it remains within a safe temperature range. Conventional surgical site temperature control involves real-time monitoring temperature. If the temperature reading reaches or exceeds a safety limit (e.g., a preset threshold), a user (e.g., a physician) can lower the laser output intensity or disable the laser output temporarily. Such manual temperature adjustment have several limitations. First, since the surgical site temperature can rise quickly especially when high laser output is used during the procedure, reducing or shutting off laser output when the temperature reading reaches or exceeds a safety limit may be too late to prevent laser-induced tissue thermal damage. Second, timing of laser output adjustment is critical for preventing tissue damage without compromising ablation or fragmentation efficiency. Manual adjustment of laser output not only puts onus on the operating physician, but may lack precision and predictability, particularly for inexperienced physicians. Third, reducing or shutting off laser output may not produce adequate and fast temperature relief at certain surgical sites or tissue anatomy. In some cases, it is not feasible to shut off or significantly reduce laser output without compromising ablation efficiency. For at least the above reasons, the present inventors have recognized an unmet need for devices and methods for automatic and more effective temperature control to prevent heat buildup at surgical site during a procedure such as a laser lithotripsy or ultrasound lithotripsy procedure.

The present document describes systems, devices, and methods for automatic control of surgical device settings based on forecasted conditions such as a prediction of future temperature at or near the surgical site. According to one embodiment, an exemplary endoscopic surgical system comprises a endoscopic surgical device controllably coupled to a medical instrument (e.g., a laser system) and configured to deliver energy (e.g., laser energy) to an anatomical target at a surgical site during a procedure; a temperature sensor to measure temperatures in a vicinity of the surgical site at different times during the procedure; and a controller circuit to generate a temperature trend or a prediction of future temperature at the surgical site using the measured temperatures at the different times. Based at least in part on the generated temperature trend or the prediction of future temperature at the surgical site, the controller circuit can adjust at least one operating parameter associated with the endoscopic surgical system to achieve or maintain substantially (e.g., ±10%, or in some embodiments, ±5%) a desired temperature at the surgical site during the procedure. This approach may advantageously prevent, or reduce the severity of, tissue thermal damage caused by excessive energy (e.g., laser energy) delivered to the tissue site.

The systems, devices, and methods according to various embodiments discussed herein improve real-time surgical site temperature control during a laser endoscopy procedure. Features described herein may further be used in regard to an endoscope, laser surgery, laser lithotripsy or ultrasound lithotripsy, irradiation parameter settings, and/or spectroscopy. Examples of targets and applications may include laser lithotripsy or ultrasound lithotripsy of renal calculi and laser incision or vaporization of soft tissue. In an example of endoscopic system that incorporate the features as described herein, surgical site temperature may be monitored and trended, and a future temperature may be predicted based on the temperature trend. Compared to conventional display of temperature measurement, prediction of future temperature can facilitate early and more effective preventive actions to be taken well before the temperature rises to a critical level, thereby preventing tissue thermal damage and improving patient safety.

The present document describes various temperature control means to regulate surgical site temperature, such as keeping the temperature below a critical level or within a desired safety range. In one example, laser output intensity or one or more laser irradiation parameters (e.g., one or more laser pulse parameters such as, power, duration, frequency, or pulse shape, exposure time, or firing angle) may be adjusted. Additionally or alternatively, irrigation inflow into the surgical site and/or outflow (suction) out of the surgical site may be regulated to put the surgical site temperature under control. In some embodiments, irrigant can be treated (e.g., chilled) before flowing into the surgical site to more quickly and effectively reduce the surgical site temperature. One or more of such temperature control means may be optimized based on surgical site conditions. For example, based on the tissue pressure at or near the surgical site, irrigation or suction flow may be adjusted to achieve or maintain a desired environment pressure at or near the surgical site while producing the temperature control effect. In some examples, multiple temperature control means (e.g., adjusting laser output or irradiation parameter, adjusting irrigation or suction flow, or providing irrigant treatment) may be combined and/or prioritized to establish a tiered temperature control strategy based on the surgical site condition. Compared to conventional approach that focuses on controlling laser output, the various temperature control means and the tiered temperature control strategy as discussed in this document advantageously allows for more versatile control of surgical site temperature in accordance with the surgical site conditions. Using alternative temperature control means such as irrigation or suction flow and irrigant treatment can help avoid discontinuation or substantial reduction of laser energy output during a laser lithotripsy procedure, such that laser therapy efficacy would not be significantly compromised. Consequently, more precise and faster temperature control and improved laser therapy efficacy and tissue safety may be achieved.

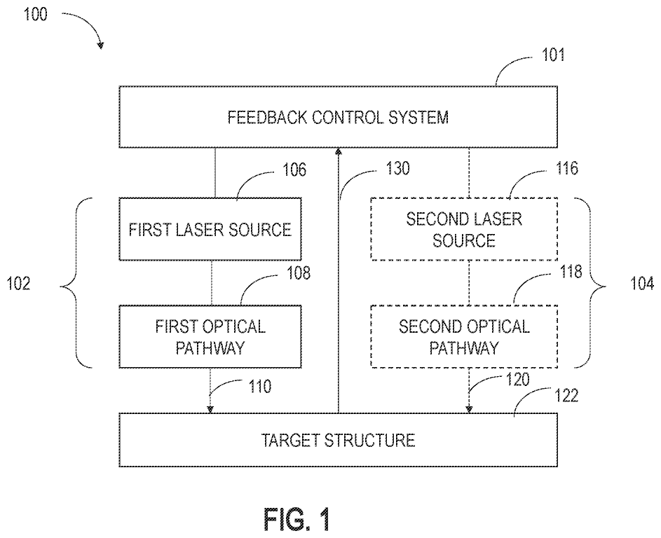

FIG. 1 is a block diagram illustrating an example of a laser energy delivery system 100 configured to provide laser treatment to an anatomical target at or near a surgical site 122 in a body of a subject, such as anatomical structure (e.g., soft tissue, hard tissue, or abnormal such as cancerous tissue) or calculi structure (e.g., kidney or pancreobiliary or gallbladder stone). In some examples, the laser energy delivery system 100 may deliver precisely controlled therapeutic treatment of tissue or other anatomical structures (e.g., tissue ablation, coagulation, vaporization, or the like) or treatment of non-anatomical structures (e.g., ablation or dusting of calculi structures).

The laser energy delivery system 100 can include a feedback control system 101, and at least one laser system 102 in operative communication with the feedback control system 101. By way of example and not limitation, FIG. 1 shows the laser feedback system connected to a first laser system 102 and optionally (shown in dotted lines) to a second laser system 104. Additional laser systems are contemplated within the scope of the present disclosure. The first laser system 102 may include a first laser source 106, and associated components such as power supply, display, cooling systems and the like. The first laser system 102 may also include a first optical pathway 108 operatively coupled with the first laser source 106. In an example, the first optical pathway 108 includes an optical fiber. The first optical pathway 108 may be configured to transmit laser beams from the first laser source 106 to the target structure at or near a surgical site 122.

The feedback control system 101 may receive feedback signals 130 from the target. Various feedback signals may be used to control laser delivery, laser energy output, and/or other system parameters to improve therapy efficacy and to achieve or maintain a desired condition such as a desired temperature at or near the surgical site to prevent or reduce the severity of laser-induced tissue thermal damage. In an example, the feedback signals 130 may include signals indicative of surgical site condition such as a temperature or a pressure at or near the surgical site during the procedure. In an example, the feedback signals 130 may include an acoustic signal produced by a laser pulse propagating through the media (e.g., liquid and vapor), projecting to the target and causing the target to vibrate. In another example, the feedback signals 130 may include reflected electromagnetic signal (e.g., reflected illumination light emitted from a light source). In yet another example, the feedback signals 130 may include reflected laser signal. The feedback control system 101 may analyze the feedback signals 130, generate signal properties from the feedback signals 130, and control laser output (e.g., energy intensity, or other laser irradiation parameters such as power, duration, frequency, or pulse shape, exposure time, or firing angle) or other system parameters according to the signal properties. In an example where the feedback signals 130 are indicative of surgical site conditions such as temperature during the procedure, the feedback control system 101 may generate a temperature trend or a prediction of future temperature at the surgical site using the feedback signals 130. Based on the temperature trend or the prediction of future temperature, the feedback control system 101 may adjust laser output or laser delivery and/or other system parameters to achieve or maintain a desired surgical site condition, such as a desired surgical site temperature during the procedure to prevent or reduce the severity of laser-induced tissue thermal damage.

As shown in FIG. 1, based on the analysis of the feedback signals 130, the feedback control system 101 may control the first laser system 102 and/or the second laser system 104 to generate suitable laser outputs to achieve a desired therapeutic effect and to achieve or maintain a desired condition such as a desired temperature at or near the surgical site to prevent or reduce the severity of laser-induced tissue thermal damage. For instance, the feedback control system 101 may monitor properties of the target structure during a therapeutic procedure (e.g., ablating calculi such as kidney stones into smaller fragments) to determine if the tissue was suitably ablated prior to another therapeutic procedure (e.g., coagulation of blood vessels).

In an example, the first laser source 106 may be configured to provide a first output 110. The first output 110 may extend over a first wavelength range, such as one that corresponds to a portion of the absorption spectrum of the target structure at the surgical site 122. The first output 110 may provide effective ablation and/or carbonation of the target structure since the first output 110 is over a wavelength range that corresponds to the absorption spectrum of the tissue.

In an example, the first laser source 106 may be configured such that the first output 110 emitted at the first wavelength range corresponds to high absorption (e.g., exceeding about 250 cm⁻¹) of the incident first output 110 by the tissue. In example aspects, the first laser source 106 may emit first output 110 between about 1900 nanometers (nm) and about 3000 nm (e.g., corresponding to high absorption by water) and/or between about 400 nm and about 520 nm (e.g., corresponding to high absorption by oxy-hemoglobin and/or deoxy-hemoglobin). Appreciably, there are two main mechanisms of light interaction with a tissue: absorption and scattering. When the absorption of a tissue is high (absorption coefficient exceeding 250 cm⁻¹) the first absorption mechanism dominates, and when the absorption is low (absorption coefficient less than 250 cm⁻¹), for example lasers at 800-1100 nm wavelength range, the scattering mechanism dominates.

Various commercially available medical-grade laser systems may be suitable for the first laser source 106. For instance, semiconductor lasers such as InXGa1−XN semiconductor lasers providing the first output 110 in the first wavelength range of about 515 nm and about 520 nm or between about 370 nm and about 493 nm may be used. Alternatively, infrared (IR) lasers such as those summarized in Table 1 below may be used.

TABLE 1
Example List of suitable IR lasers

| | | | Optical |
|---|---|---|---|
| | | Absorption | Penetration |
| | Wavelength | Coefficient | Depth |
| Laser | λ (mm) | μ₂ (cm⁻¹) | δ (μm) |
| Thulium fiber laser: | 1908 | 88/150 | 114/67 |
| Thulium fiber laser: | 1940 | 120/135 | 83/75 |
| Thulium:YAG: | 2010 | 62/60 | 161/167 |
| Holmium:YAG: | 2120 | 24/24 | 417/417 |
| Erbium:YAG: | 2940 | 12,000/1,000 | 1/10 |

The optional second laser system 104 may include a second laser source 116 for providing a second output 120, and associated components, such as power supply, display, cooling systems and the like. The second laser system 104 may either be operatively separated from or, in the alternative, operatively coupled to the first laser source 106. In some embodiments, the second laser system 104 may include a second optical pathway 118 (separate from the first optical pathway 108) operatively coupled to the second laser source 116 for transmitting the second output 120. Alternatively, the first optical pathway 108 may be configured to transmit both the first output 110 and the second output 120.

In certain aspects, the second output 120 may extend over a second wavelength range, distinct from the first wavelength range. Accordingly, there may not be any overlap between the first wavelength range and the second wavelength range. Alternatively, the first wavelength range and the second wavelength range may have at least a partial overlap with each other. In advantageous aspects of the present disclosure, the second wavelength range may not correspond to portions of the absorption spectrum of the target structure where incident radiation is strongly absorbed by tissue that has not been previously ablated or carbonized. In some such aspects, the second output 120 may advantageously not ablate uncarbonized tissue. In another embodiment, the second output 120 may ablate carbonized tissue that has been previously ablated. In additional embodiments, the second output 120 may provide additional therapeutic effects. For instance, the second output 120 may be more suitable for coagulating tissue or blood vessels.

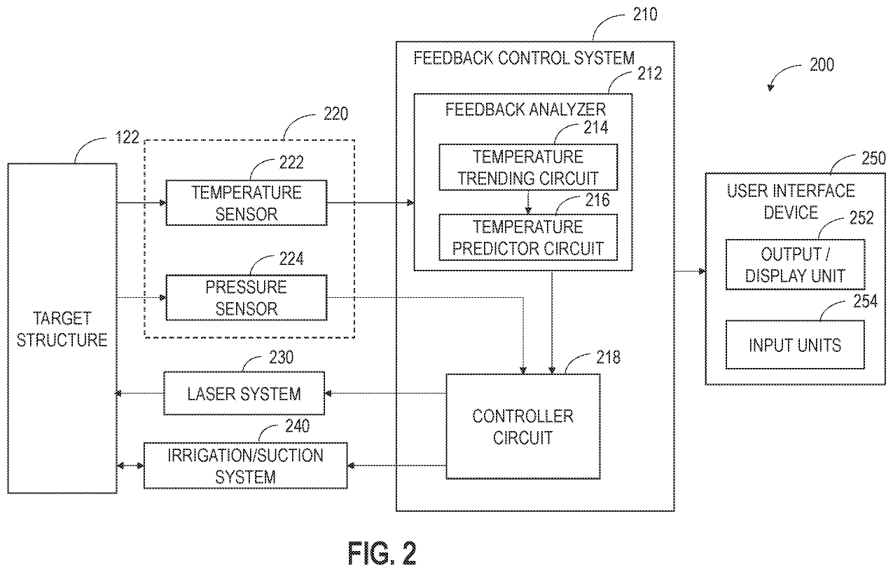

FIG. 2 is a block diagram illustrating an endoscopic surgical system 200 with automatic surgical site condition control, and at least a portion of the environment in which the system 200 may operate. The system 200 can be an embodiment of the laser energy delivery system 100, or a lithotripsy system that may be used for destructing hardened masses like kidney stones, bezoars, gallstone, among other calculi structures. The system 200 may monitor and control conditions of the surgical site 122 during a laser procedure, such as to keep the surgical site temperature at substantially a desired level during a procedure to prevent or reduce the severity of laser-induced tissue thermal damage.

The endoscopic surgical system 200 can includes a feedback control system 210, one or more sensors 220, a laser system 230, an irrigation and/or suction system 240, and a user interface device 250. The feedback control system 210, which is an embodiment of the feedback control system 101 of the laser energy delivery system 100, can include a feedback analyzer 212 and a controller circuit 218. According to example embodiments, the feedback control system 210 may include processors, such as microprocessors, digital signal processors (DSPs), application specific integrated circuits (ASICs), field programmable gate arrays (FPGAs), or any other equivalent integrated or discrete logic circuitry, as well as any combinations of such components for performing one or more of the functions attributed to the feedback control system 210. The feedback analyzer 212 may be communicatively coupled to one or more sensors 220, receive therefrom feedback signals, and analyze the feedback signals to generate one or more signal properties that may be used for controlling surgical site conditions. In an example as illustrated in FIG. 2, the one or more sensors 220 can include a temperature sensor 222 configured to sense a temperature in a vicinity of the surgical site during a laser procedure. Examples of the temperature sensor 222 can include a thermocouple, a thermistor, a resistance temperature detector, or a semiconductor-based sensor incorporated in integrated circuits (IC). The temperature sensor 222 can be positioned at a distal portion of a laser delivery system (e.g., distal end of an optical pathway or a laser fiber for directing the laser energy to the surgical site), or a distal portion of an endoscope, to expose the temperature sensor in the environment of the surgical site and directly measure the temperature therein. The one or more sensors 220 can additionally include a pressure sensor 224 to sense surgical site pressure during the procedure.

The feedback analyzer 212 can include one or more of a temperature trending circuit 214 or a temperature predictor circuit 216. The temperature trending circuit 214 can generate a trend of temperature measurements over time. Based on the temperature trend, the feedback analyzer 212 can determine a temperature change rate (ΔT/Δt) indicating an amount of change in temperature (ΔT) over a unit time period (Δt), such as a temperature rising rate at or near the surgical site.

In some examples, the temperature sensor 222 can be positioned at a location away from the surgical site, such as at portion of the irrigation and/or suction system 240, to measure an “offsite” temperature not equivalent to but correlated with the surgical site temperature. The feedback analyzer 212 can estimate the surgical site temperature using an trained estimation model and the measured offsite temperature. In some examples, the estimation of the surgical site temperature may be further based on other conditions such as laser energy output settings (e.g., power, pulse width, pulse frequency) of the laser system 230 and irrigation inflow and outflow settings of the irrigation and/or suction system 240. U.S. patent application Ser. No. 15/686,465, entitled “Automatic Irrigation-Coordinated Lithotripsy,” refers to devices and methods for estimating a temperature at the surgical place using various factors including, for example, irrigation fluid temperature, irrigation volume, and energy applied to the surgical place. The entirety of such reference is incorporated herein by reference.

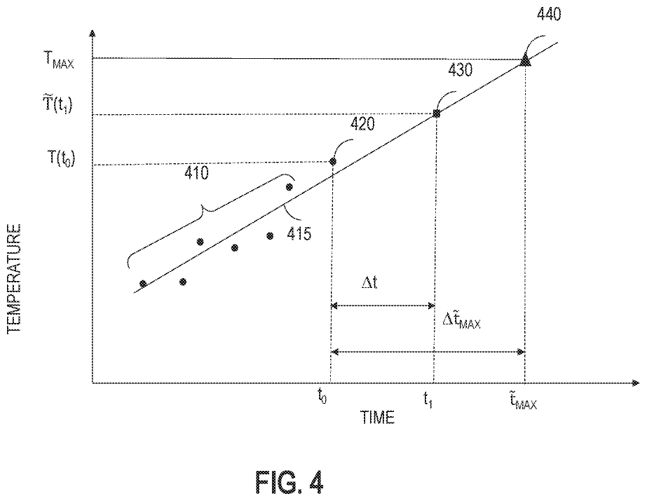

The temperature predictor circuit 216 can generate a prediction of future temperature at the surgical site at a specified future time based on surgical site temperatures measured at different times, under the assumption that the laser energy applied to the surgical site and the heat dissipation mechanisms (e.g., natural, or artificially applied such as via irrigation flow or other means of surgical site temperature control) remain unchanged. In an example, the temperature predictor circuit 216 may generate a prediction model using the plurality of temperature measurements, and generate a prediction of future temperature using the prediction model. The prediction model can be generated using techniques such as curve and surface fitting, time series regression, or machine learning (ML) approaches. The prediction model can be generated through a model training process using a training dataset including surgical site temperatures measured at different times and a model type. The training process includes algorithmically adjusting model parameters (e.g., weights assigned to nodes of an input layer, output layer, or any hidden layers of a neural network model) until a convergence criteria or a training stopping criterion is satisfied. The prediction model can include a linear model or a non-linear model, in which the surgical site temperature can be modeled to have a linear relationship or a nonlinear relationship with time. In an example, as illustrated in FIG. 2, the temperature predictor circuit 216 may generate a prediction of future surgical site temperature using the temperature trend, or the temperature change rate, generated by the temperature trending circuit 214. FIG. 4 illustrates an example of a temperature trend 415 and a prediction of a future temperature using the trend. In this example, the temperature trend 415, such as generated by the temperature trending circuit 214, is a linear trend generated using a regression analysis of past temperature measurements (represented by data points 410) at different times and a current temperature T(t₀) at time t₀ (represented by data points 420), such as measured using the temperature sensor 222. The linear temperature trend 415 can be characterized by a temperature change rate, represented by the slope of the linear trend 415. For example, the temperature trending circuit 214 may analyze the temperatures measurements that have increased at 3° C. over the previous 6 seconds prior to the present measurement, and determines a temperature change rate of +0.5° C. per second (+0.5° C./sec, where “+” indicates a temperature rise). Based on the current temperature T(t₀) (e.g., 40° C.) and the temperature increase rate (e.g., +0.5° C./sec), the temperature predictor circuit 216 can predict a future temperature \tilde{T}(t₁), represented by data point 430, at a future time t₁ (t₁=t₀+Δt). For example, it can be predicted that in next 10 seconds (Δt=10 seconds), the predicted surgical site temperature \tilde{T}(t₁) can reach up to 45° C. (40° C.+0.5° C./sec*10 sec).

In some examples, when the measured temperature is below a pre-determined upper “safe-operating” temperature limit (max temperature, or Tₘₐₓ), the temperature predictor circuit 216 may estimate a safe-operation time window representing time taken for the temperature to reach or exceed the “safe-operating” temperature limit Tₘₐₓ. As illustrated in FIG. 4, the safe-operation time window Δ\tilde{t}ₘₐₓ represents an estimated time lag from current temperature measurement T(t₁) at time t₁ (illustrated as data point 420) to the “safe-operating” temperature limit Tₘₐₓ at an estimated time \tilde{t}ₘₐₓ (illustrated as data point 440). The Δ\tilde{t}ₘₐₓ can be estimated based on the temperature trend 415. For example, for a “safe-operating” temperature limit of Tₘₐₓ=60° C., the temperature predictor circuit 216 can predict that the surgical site temperature will rise from current T(t₁) of 40° C. to Tₘₐₓ of 60° C. in next Δtₘₐₓ=(60° C.−40° C.)/(0.5° C./sec)=40 seconds. Information about the temperature trend 415, the prediction of future temperatures, or the safe-operation time window Δ\tilde{t}ₘₐₓ may be presented to user, such as via the user interface device 250. The user may be alerted about temperature rise at the surgical site, and recommended to take proper preventive actions (e.g., adjusting laser output or other system parameters) well before the temperature reaches the “safe-operating” temperature limit, thereby preventing tissue thermal damage and improving patient safety during the procedure.

The controller circuit 218 may be coupled by wired or wireless connections to the feedback analyzer 212. The controller circuit 218 can compare the monitored surgical site temperature against a specific temperature range, such as the upper “safe-operating” temperature limit as previously described. When the measured surgical site temperature is within the specific temperature range (e.g., below the “safe-operating” temperature limit), the controller circuit 218 may adjust one or more system parameters, such as laser energy output via the laser system 230 and/or irrigation flow via the irrigation and/or suction system 240, in accordance with one or more of the temperature trend (or the temperature change rate) generated by the temperature trending circuit 214, or the prediction of future temperature or the estimated safe-operation time window generated by the temperature predictor circuit 216. For example, if the surgical site temperature satisfies a criterion that warrants temperature adjustment (e.g., if the temperature rising rate (e.g., +0.8° C./sec) exceeds a rate threshold (e.g., +0.5° C./sec), or if the prediction of future temperate in next X seconds (e.g., 15 seconds) would exceed the “safe-operating” temperature limit, or if the estimated safe-operation time window (e.g., 10 seconds for the surgical site temperature to rise to the “safe-operating” temperature limit) is shorter than a pre-determined threshold window length), then the controller circuit 218 can automatically, or prompt the user to manually, adjust one or more system parameters to prevent or reduce the severity of laser-induced tissue thermal damage, as to be further discussed below.

The laser system 230, which is an example of the laser system 102 or the laser system 104 as shown in FIG. 1, can include a laser source (such as the first laser source 106) and an optical pathway (such as the first optical pathway 108) for directing the laser energy to the surgical site. The laser source can generate laser energy in accordance with a laser output intensity or one or more laser irradiation parameters (e.g., one or more laser pulse parameters such as, power, duration, frequency, or pulse shape, exposure time, or firing angle). At least some of the laser parameters are programmable or adjustable either automatically such as by the controller circuit 218, or manually by a user via the user interface device 250. When the measured surgical site temperature is within the specific temperature range (e.g., below the “safe-operating” temperature limit), the controller circuit 218 can automatically adjust a laser output setting in accordance with one or more of the temperature trend (or the temperature change rate) generated by the temperature trending circuit 214, or the prediction of future temperature or the estimated safe-operation time window generated by the temperature predictor circuit 216. For example, when the surgical site temperature satisfies a temperature adjustment criterion (the temperature rising rate exceeds a rate threshold, or if the prediction of future temperate in next X seconds would exceed the “safe-operating” temperature limit, or if the estimated safe-operation time window is shorter than a pre-determined threshold window length), the controller circuit 218 can automatically reduce average power of laser pulses delivered to the surgical site, such as by reducing one or more of a pulse width of a laser pulse, a peak power of a laser pulse, or a pulse frequency representing a number of laser pulses per unit time. Reducing the average power of laser pulses can decrease the laser-induced heating effect at or near the surgical site, thereby preventing tissue thermal damage and improving patient safety during the procedure.

In addition or alternative to adjusting one or more laser output parameters, the controller circuit 218 can automatically select one of a plurality of pre-determined laser output settings or pulse profiles with different energy output levels based on one or more of the temperature trend (or the temperature change rate) generated by the temperature trending circuit 214, or the prediction of future temperature or the estimated safe-operation time window generated by the temperature predictor circuit 216. In an example, the controller circuit 218 can automatically toggle between at least a first “high output” setting and a second “low output” setting with respective pre-determined parameter values. The “low output” setting has a lower average power than the “high output” setting. When the surgical site temperature satisfies the temperature adjustment criterion, the laser output setting can be automatically switched to “low output” setting.

In some examples, when the measured surgical site temperature is within the specific temperature range (e.g., below the “safe-operating” temperature limit), the controller circuit 218 can generate a control signal to an actuator coupled to an optical pathway (e.g., a laser fiber) of the laser system 230 to adjust the position or orientation of a distal portion (laser firing portion) of the optical pathway relative to the anatomical target at or near the surgical site. The adjustment of the position or orientation of the distal portion of the optical pathway can include adjusting a distance between the distal portion and the anatomical target (the “fiber-target” distance), or an aiming angle of the distal portion with respect to the anatomical target, in accordance with one or more of the temperature trend (or the temperature change rate) generated by the temperature trending circuit 214, or the prediction of future temperature or the estimated safe-operation time window generated by the temperature predictor circuit 216. For example, when the surgical site temperature satisfies a temperature adjustment criterion (e.g., the temperature rising rate exceeds a rate threshold, the prediction of future temperate in next X seconds would exceed the “safe-operating” temperature limit, or the estimated safe-operation time window is shorter than a pre-determined threshold window length), the controller circuit 218 can automatically, via the actuator, move the distal portion of the optical pathway farther away from the surgical site (i.e., increase the fiber-target distance) and/or rotating the distal portion of the optical pathway to aim the laser away from the surgical site (to increase the aiming angle). By increasing the fiber-target distance and/or increasing the aiming angle, the density of the laser energy incident on the surgical site and the laser-induced heat transferred into the surgical site can be reduced.

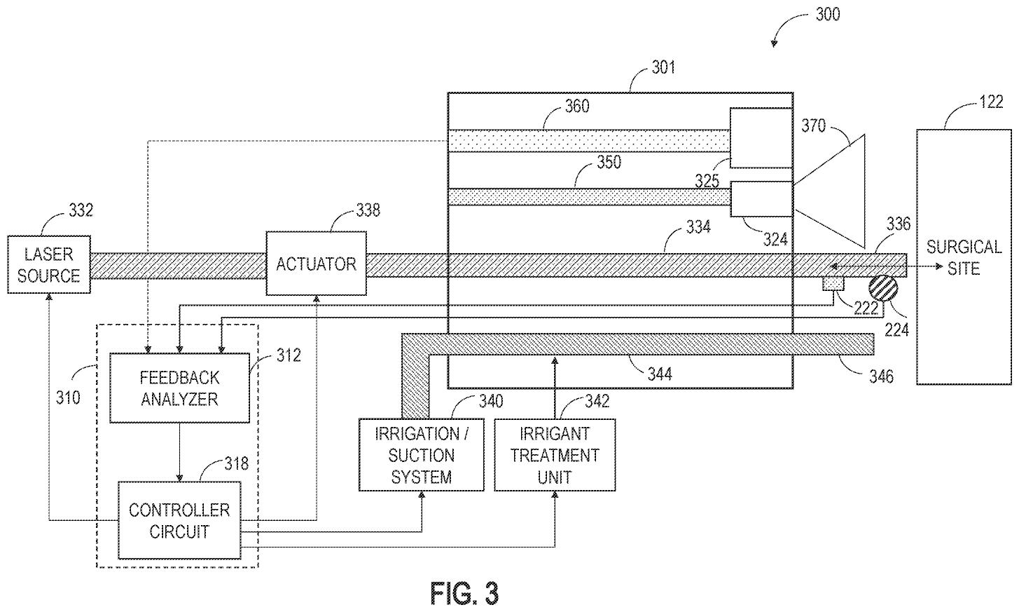

The irrigation and/or suction system 240 can include one or more irrigation and/or suction sources that can provide a flow of irrigation fluid (also referred to as irrigant, e.g., saline solution) to the surgical site through at least one irrigation channel such as included in an endoscope during the procedure. The irrigation fluid can facilitate removal of the tissue debris, stone fragments, and other unwanted matters through a suction channel. The irrigation flow also has a cooling effect on the tissue at or near the surgical site and the surgical tools (e.g., endoscopic tissue removal device), and can help dissipate the heat generated during ablation of calculi. Examples of the irrigation and/or suction system 240 are discussed below with reference to FIG. 3.

When the measured surgical site temperature is within the specific temperature range (e.g., below the “safe-operating” temperature limit), the controller circuit 218 can automatically adjust one or more irrigation parameters, such as an irrigation flow or a suction flow, in accordance with one or more of the temperature trend (or the temperature change rate) generated by the temperature trending circuit 214, or the prediction of future temperature or the estimated safe-operation time window generated by the temperature predictor circuit 216. For example, when the surgical site temperature satisfies a temperature adjustment criterion (e.g., the temperature rising rate exceeds a rate threshold, or the prediction of future temperate in next X seconds would exceed the “safe-operating” temperature limit, or the estimated safe-operation time window is shorter than a pre-determined threshold window length), the controller circuit 218 can automatically increase the irrigation flow from the irrigation source to the surgical site to increase convective heat transfer. Additionally or alternatively, the controller circuit 218 can automatically increase the suction flow (or suction pressure) to more effectively withdraw the fluid away from the surgical site to improve heat dissipation and reduce the surgical site temperature.

The application of irrigation or suction flow for controlling surgical site temperature can fluctuate the pressure at or near the surgical site. For example, irrigation flow into the surgical site would generally increase the surgical site pressure (positive pressure change), while a suction pressure (i.e., outflow) would generally decrease the surgical site pressure (negative pressure change). Such irrigation and/or suction-induced positive or negative pressure changes, if not properly regulated, may be harmful to tissue or organs at or near the surgical site. To keep the pressure of the anatomical environment under control during the procedure and to avoid or reduce pressure-related tissue damage, the system 200 can include a pressure sensor 224 to sense surgical site pressure during the procedure. When the surgical site temperature satisfies a temperature adjustment criterion (e.g., the temperature rising rate exceeds a rate threshold, the predicted future temperate in next X seconds would exceed the “safe-operating” temperature limit, or the estimated safe-operation time window is shorter than a pre-determined threshold window length), the controller circuit 218 can selectively activate or adjust irrigation flow or the suction flow based on the measured surgical site pressure (P). For example, as an increase in irrigation flow into the surgical site may introduce a positive pressure change at or near the surgical site, if the measured surgical site pressure, P, exceeds a pre-determine or user-specified upper pressure limit Pₘₐₓ (P>Pₘₐₓ), then the controller circuit 218 can increase the suction flow to reduce the surgical site temperature, but avoid increasing the irrigation flow to prevent further increase in surgical site pressure. For example, the irrigation flow can be maintained at its current rate or set to a reduced rate, or temporarily deactivated. The increased suction flow may also help reduce the surgical site pressure to a level within the desired pressure range. If the measured surgical site pressure is within a desired pressure range between the upper pressure limit Pₘₐₓ and a lower pressure limit Pₘᵢₙ (Pₘᵢₙ<P<Pₘₐₓ), then the controller circuit 218 can increase one or both of the irrigation flow and the suction flow to reduce the surgical site temperature. As an increase in suction flow may introduce a negative pressure change at or near the surgical site, if the measured surgical site pressure falls below the lower pressure limit Pₘᵢₙ (P<Pₘᵢₙ), then the controller circuit 218 can increase the irrigation flow into the surgical site to reduce the surgical site temperature, but avoid increasing the suction flow to prevent further decrease in surgical site pressure. For example, the suction flow can be maintained at its current rate or set to a reduced rate, or temporarily deactivated. The increased irrigation flow may also help increase the surgical site pressure to a level within the desired pressure range.

In some examples, the irrigation and/or suction system 240 can include an irrigant treatment unit that can adjust the temperature of the irrigation fluid (irrigant) before being applied to the surgical site. When the measured surgical site temperature is within the specific temperature range (e.g., below the “safe-operating” temperature limit), the controller circuit 218 can generate a control signal to the irrigant treatment unit to alter the temperature of the irrigant in accordance with one or more of the temperature trend (or the temperature change rate) generated by the temperature trending circuit 214, or the prediction of future temperature or the estimated safe-operation time window generated by the temperature predictor circuit 216. In an example, the irrigant treatment unit can include a cooling system (e.g., a radiator, or an in-line chiller). When the surgical site temperature satisfies a temperature adjustment criterion (e.g., the temperature rising rate exceeds a rate threshold, the predicted future temperate in next X seconds would exceed the “safe-operating” temperature limit, or the estimated safe-operation time window is shorter than a pre-determined threshold window length), the cooling system can, under the control of the controller circuit 218, cool the irrigant before reaching the surgical site. In another example, the irrigant treatment unit includes a fluid mixer. If the temperature rising rate exceeds a rate threshold, or if the prediction of future temperate in next X seconds would exceed the “safe-operating” temperature limit, or if the estimated safe-operation time window is shorter than a pre-determined threshold window length, the fluid mixer can, under the control of the controller circuit 218, mix at least two irrigant sources of different temperatures before reaching the surgical site. The cooled irrigate via the cooling system or the mixed irrigant via the fluid mixer, when applied to the surgical site can improve convective heat transfer therein and effectively and efficiently reduce the surgical site temperature.

In some examples, the controller circuit 218 can maintain the surgical site temperature at substantially a desired level or range in accordance with a temperature management plan. The temperature management plan can include a prioritized order of two or more temperature control means described above, including, for example, changing a laser output setting or one or more laser irradiation parameters, adjusting the position or orientation of the distal portion of the optical pathway (e.g., a laser fiber), activating or adjusting an irrigation flow into the surgical site and/or a suction flow away from the surgical site, or altering the temperature of the irrigant before being applied to the surgical site, among other means. The temperature management plan can be programmed or modified by a user, such as via the user interface device 250. The order of the temperature control means can be determined based on the availability (e.g., irrigant cooling system), efficiency of temperature control, or potential adverse effects on the surgical site. In an example, the temperature management plan may be programmed with a bias toward maintaining an optimal or a user-selected laser output setting, while adjusting other device settings (e.g., position or orientation of the distal portion of the optical pathway, irrigation and/or suction flows, irrigant temperature) suitable for managing surgical site temperature. Maintaining the laser output setting can be desirable during a laser lithotripsy procedure to reduce procedure time and ensure therapy efficacy and efficiency. Additionally, adjusting laser output setting (e.g., reducing average laser power) may have a slow effect on the surgical site temperature. In some examples, a lithotripsy system may be biased toward maintaining a physician selected peak power setting of the laser, even when adjusting the laser output setting. Laser pulse energy can be related to pulse width and peak power according to Equation (1) below:

Pulse Energy=Pulse Width*Peak Power (1)

Thus, in some instances the pulse energy may be decreased by decreasing the pulse width without lowering the peak power. Additionally, an average laser power can be related to the pulse energy and pulse frequency (i.e., the number of pulses per second) according to Equation (2) below:

Average Power=Pulse Energy*Pulse Frequency (2)

Since the fluid temperature increases proportionally to the laser average power, reducing pulse energy and maintaining the same pulse frequency can reduce the average power delivered by the laser to the anatomic site. Combining Equations (1) and (2) yields Equation (3) below:

Average Power=Pulse Width*Peak Power*Pulse Frequency (3)

The Equation (3) defines the relationship between the three laser variables (pulse width, peak power, and pulse frequency). Said laser variables may be individually or collectively adjusted to produce a desired average power (heating potential) of the laser emission delivered to an anatomic site. For example, in some instances, the average power may be decreased by decreasing pulse frequency and keeping both peak power and pulse width constant.

In some examples, to prevent irrigation and/or suction-induced pressure fluctuation at or near the surgical site, the temperature management plan may be programmed such that irrigant temperature control (e.g., chill the irrigant before being applied to the surgical site) can be used before an attempt to adjust the irrigation or suction flow. For example, when the measured surgical site temperature satisfies a temperature adjustment criterion (e.g., the temperature rising rate exceeds a rate threshold, the predicted future temperate in next X seconds would exceed the “safe-operating” temperature limit, or the estimated safe-operation time window is shorter than a pre-determined threshold window length), the controller circuit 218 may first generate a control signal to the irrigant treatment unit of the irrigation and/or suction system 240 to chill the irrigant before being applied to the surgical site. The feedback control system 210 may then re-evaluate the surgical site temperature to determine if it still satisfies a temperature adjustment criterion; and if so, the controller circuit 218 may generate a control signal to the irrigation and/or suction system 240 to increase the irrigation flow and/or the suction flow to reduce the surgical site temperature. The choice between, or the order of applying, irrigation flow and suction flow can be based on the surgical site pressure, as discussed above.

For example, to keep the surgical site temperature from reaching or exceeding the “safe-operating” temperature limit Tₘₐₓ, the system may compare the current surgical site pressure, P, to the pre-determine or user-specified upper pressure limit Pₘₐₓ. If the current surgical site pressure P is substantially below Pₘₐₓ (e.g., the difference between P and Pₘₐₓ exceeds a threshold), then an irrigation inflow rate may be increased to increase convective heat transfer via irrigation. Additionally or alternatively, suction flow can be increased to more efficiently take the heat away from the surgical site. In contrast, if the current surgical site pressure P is substantially close to Pₘₐₓ (e.g., within a user-specified or pre-determined margin, e.g., ±10%), then rather than increasing the irrigation inflow rate, the laser output setting may be lowered. In embodiments where suction flow is actively controlled (e.g., via a pump), if the current surgical site pressure P is substantially close to Pₘₐₓ, then the suction flow rate can be increased to decrease the surgical site pressure in lieu of, or in addition to, lowering the laser output setting. While the body tissue can generally regulate some positive pressure changes, many organs are relatively defenseless to negative pressure changes. Accordingly, in some examples, increasing the irrigation flow may be attempted prior to activating or increasing the suction flow.

The feedback control system 210 may then re-evaluate the surgical site temperature to determine if it still satisfies a temperature adjustment criterion; and if so, the controller circuit 218 may generate a control signal to the laser system 230 to adjust the position or orientation of the distal portion of the optical pathway, or to change a laser output setting or one or more laser irradiation parameters. The tiered, sequential activation or adjustment of different temperature control means can help achieve or maintain a desired surgical site condition (e.g., temperature, pressure) during the procedure without comprising therapy efficacy and efficiency or imposing additional risk of tissue damage at or near the surgical site.

The user interface device 250 may be operatively in communication with the feedback control system. The user interface device 250 can include an output/display unit 252 to display information including, for example, surgical site conditions such as the temperature, pressure, or other information sensed by the sensors 220, feedback signal generated by the feedback analyzer 212 including the temperature trend, the prediction of future temperatures, or the safe-operation time window (time taken for the temperature to reach or exceed the “safe-operating” temperature limit), or current device settings such as the laser output setting or irrigation or suction flow rates, etc. The output/display unit 252 can display UI elements including visual elements, alerts, tactile feedback, or any combination thereof. The output/display unit 252 can generate an alert about potentially hazardous condition at or near the surgical site, such as an elevated temperature satisfying a temperature adjustment criterion that warrants preventive temperature adjustment, or an elevated surgical site pressure. The alert can be presented in an audible, visible, tactile, or otherwise human-perceptible format. In an examples, the output/display unit 252 can display a countdown timer, a progress bar, or other UI element to graphically and/or textually represent the safe-operation time window, and recommend the user to adjust one or more system parameters (e.g., lowering the laser output or increasing irrigation and/or suction) to prolong the time before the “safe-operating” temperature limit Tₘₐₓ is reached. For example, if the temperature predictor circuit 216 predicts that the surgical site temperature will reach the “safe-operating” temperature limit Tₘₐₓ in 30 seconds, then the output/display unit 252 may display a countdown timer, and/or a recommendation to the user to lower the laser setting or take other temperature control means as described above to prolong the time until the Tₘₐₓ is reached.

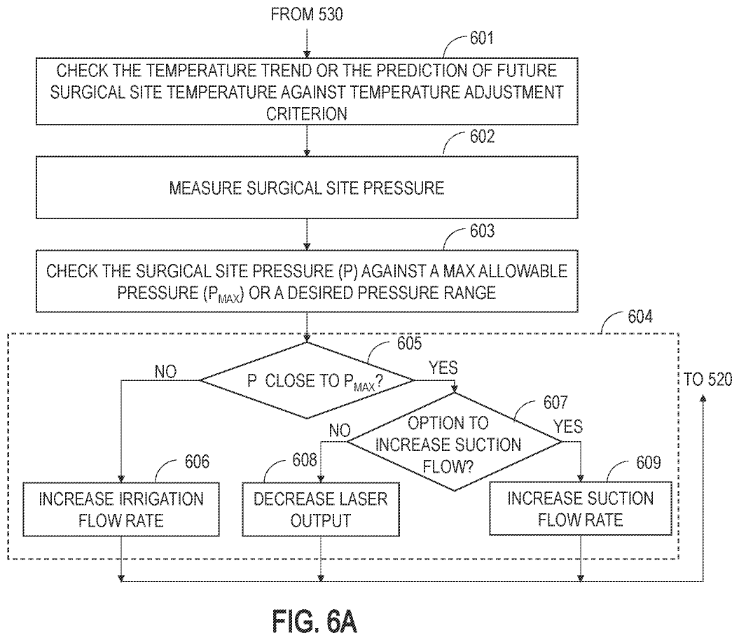

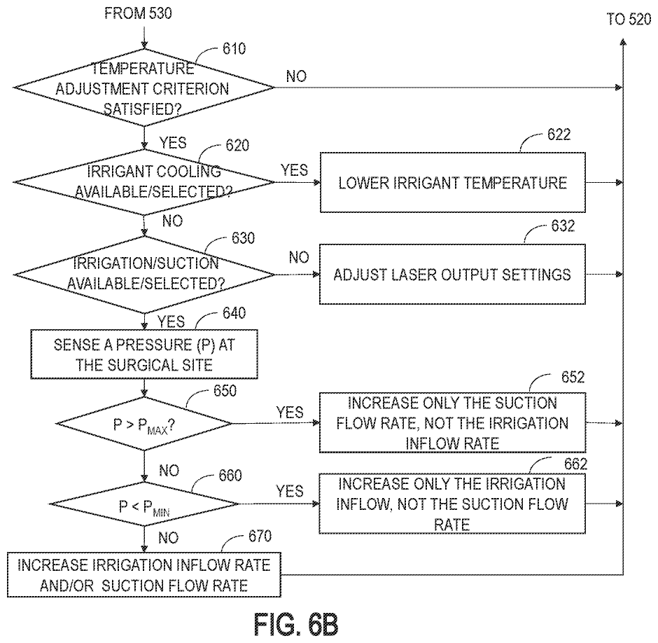

The user interface device 250 can include one or more input units 254 to receive user programming of the device, such as parameter values that define the temperature adjustment criterion (the temperature rising rate threshold, the “safe-operating” temperature limit, or a threshold window length for the safe-operation time window), and user input to adjust laser output setting, irrigation or suction flow parameters, among other device parameters for controlling surgical site temperature. In some examples, a user may provide, via one or more input units 254, the temperature management plan that defines a prioritized order of two or more temperature control means as described above. For example, a user may guide the controller circuit 218 to first decrease irrigant temperature (if available) without adjusting laser output of irrigation flow rate. Then, if temperature is predicted to achieve a threshold temperature (Tₜₕ) within next (Δt), then the irrigation flow rate and/or the suction flow rate can be increased, and/or the laser output can be decreased. Both Tₜₕ and Δt can be defined by the user via the one or more input units 254. In an example, as illustrated in FIG. 4, Tₜₕ can be chosen as Tₘₐₓ, and Δt can correspond to the safe-operation time window Δ\tilde{t}ₘₐₓ, Examples of prioritized means for controlling surgical site temperature are discussed below with reference to FIGS. 6A-6B.

In some examples, the output/display unit 252 may generate a recommendation for taking preventive actions to prevent tissue damage, such as recommended adjustment of laser output or other system parameters. A user may provide an input via the one or more input units 254 to confirm, reject, or modify the recommended adjustment.

FIG. 3 illustrate an example of an endoscopic laser lithotripsy system 300 with automatic surgical site condition control, which can be an example of the endoscopic surgical system 200. The endoscopic laser lithotripsy system 300 may include an endoscope 301, a feedback control system 310, an actuator 338, an irrigation and/or suction system 340, an irrigant treatment unit 342. The endoscope 301 has a proximal portion and an elongate distal portion configured to be inserted into a surgical site of a patient during an endoscopic laser lithotripsy procedure. The endoscope 301 may provide visual inspection or treatment of soft (e.g., non-calcified) or hard (e.g., calcified) tissue as well as for visualizing or breaking up or otherwise treating kidney stones or other stones or other targets. As illustrated in FIG. 3, the endoscope 301 may include or provide visualization and illumination optics, such as a visualization optical pathway 360 and an illumination optical pathway 350, each of which may extend longitudinally along the elongate body of the endoscope 301. An eyepiece or camera or imaging display may be provided at or coupled to the visualization optical pathway 360 to permit user or machine visualization of a target region at or near a distal end of the endoscope 301. The target region may be illuminated by light 370, such as provided by an illumination light source 324 at a proximal end of the illumination optical pathway 350 and emitted from a distal end of the illumination optical pathway 350. The light source 324 can include, for example, a Xenon lamp, a light-emitting diode (LED), a laser diode (LD), or any combination thereof. In an example, the light source 324 may include two or more light sources that emit light having different illumination characteristics, referred to as illumination modes. In an example, the illumination modes may include a white light illumination mode, or a special light illumination mode such as a narrow band imaging mode, an auto fluorescence imaging mode or an infrared imaging mode. A special light illumination can concentrate and intensify specific wavelengths of light, for example, resulting in a better visualization of tissue or other structures at the surgical site.

The lithotripsy system 300 may include or be coupled to at least one laser source 332, which may be an example of the first laser source 106, the second laser source 116, or the laser source included in the laser system 230. The laser source 332 may be mechanically and optically connected to an optical pathway 334, which may include a single optical fiber or a bundle of optical fibers. The optical pathway 334, which is an embodiment of the first optical pathway 108 or the second optical pathway 118, or the optical pathway included in the laser system 230, may be introduced via a proximal access port to extend within a working channel or other longitudinal passage or lumen of the endoscope 301 or similar instrument.

The lithotripsy system 300 may include one or more sensors to sense information from the anatomical target or the surgical site, such as a temperature sensor 222 and a pressure sensor 224. As described above with reference to FIG. 2, the temperature sensor 222 can sense a surgical site temperature, and the pressure sensor 224 can sense a surgical site pressure, during the procedure. The temperature sensor 222 and the pressure sensor 224 can be located at a distal end 336 of the optical pathway 334. Alternatively, the temperature sensor 222 and the pressure sensor 224 may be located at other locations, such as a distal end 346 of an irrigation and/or suction channel 344. In some examples, the temperature sensor 222 and the pressure sensor 224 may be associated with different device components. For example, the temperature sensor 222 can be located at a distal end 336 of the optical pathway 334, and the pressure sensor 224 can be located at a distal end of the irrigation and/or suction channel 344, or vice versa.

The irrigation and/or suction system 340 (an embodiment of the irrigation and/or suction system 240) can include an irrigation source and a suction source, each fluidly coupled to a working channel of the endoscope 301, such as an irrigation and/or suction channel 344. The irrigation and/or suction channel 344 can be a common, unified channel for conducting irrigation inflow and suction outflow at different times. Alternatively, in some examples, the irrigation and/or suction channel 344 can comprise two separate channels, such as an irrigation channel and a suction channel. The separate irrigation channel and the suction channel may be parallel to each other, or coaxially disposed with a common axis, such as in a nested configuration. The irrigation source may function to provide irrigation fluid (irrigant) to the irrigation and/or suction channel 344. The irrigation fluid may be gravity fed or pressurized. In an example, a pump may produce pressurized irrigation flow through the irrigation and/or suction channel 344 into the surgical site. The suction source may function to pull, suck, draw, aspirate, or otherwise move or remove fluid and unwanted matters from the surgical site to a receptacle. The suction source may perform the aforementioned functions by generating and applying vacuum, suction, or negative pressure to the irrigation and/or suction channel 344.

The feedback control system 310 can receive feedback information produced by one or more sensors, including, for example, surgical site temperature measurements generated by the temperature sensor 222, and surgical site pressure measurements generated by the pressure sensor 224. The feedback control system can include a feedback analyzer 312 and a controller circuit 318. The feedback analyzer 312 (an embodiment of the feedback analyzer 212) can analyze the temperature measurements produced by the temperature sensor 222, and generate one or more temperature metrics such as a temperature rising rate, a prediction of future temperate in next X seconds, or an estimated safe-operation time window. The controller circuit 318 (an embodiment of the controller circuit 218) can determine whether the monitored surgical site temperature falls within a specific temperature range (e.g., below an upper “safe-operating” temperature limit), and satisfies a temperature adjustment criterion indicating the rise in temperature at or near the surgical site warrants temperature adjustment to prevent tissue thermal damage (e.g., the temperature rising rate exceeds a rate threshold, or if the prediction of future temperate in next X seconds would exceed the “safe-operating” temperature limit, or if the estimated safe-operation time window is shorter than a pre-determined threshold window length), as described above with reference to FIG. 2. If the temperature adjustment criterion is met, the controller circuit 318 can automatically, or prompt the user to manually, adjust one or more system parameters to regulate the surgical site temperature to prevent or reduce the severity of laser-induced tissue thermal damage.

Various temperature control means can be used to regulate surgical site temperature during a procedure. In an example, the controller circuit 318 can generate a control signal to the laser source 332 to automatically adjust a laser output setting, such as average power of laser pulses delivered to the surgical site, such as by reducing one or more of a pulse width of a laser pulse, a peak power of a laser pulse, or a pulse frequency representing a number of laser pulses per unit time. In another example, the controller circuit 318 can generate a control signal to an actuator 338 to adjust a position of a laser emitting end relative to the target at the surgical site. The actuator 338 can be coupled to a portion of the optical pathway 334, and can be in electrical communication with the controller circuit 318. In an example, the actuator 338 may be located at or near the distal end of the endoscope 301. The actuator 338 may include one or more of an electromagnetic element, an electrostatic element, a piezoelectric element, or other actuating element such as to actuate or otherwise permit longitudinal or rotational positioning of the distal end 336 of the optical pathway 334 with respect to the working channel or other longitudinal passage of the endoscope 301, or with respect to another reference location for which the endoscope 301 may serve as a frame of reference. In response to the control signal from the controller circuit 318, the actuator 338 can adjust the position or orientation of a distal end 336 of the optical pathway 334, such as adjusting the longitudinal position by moving the distal end 336 farther away from the surgical site (to increase the fiber-target distance), and/or adjusting the rotational position by steering the distal end 336 away from the surgical site (to increase the aiming angle).

In yet another example, the controller circuit 318 can generate a control signal to the irrigation and/or suction system 340 to automatically adjust one or more irrigation parameters, such as an irrigation flow or a suction flow. The irrigation flow or suction flow can help dissipate the heat generated during the procedure (e.g., laser treatment of tissue or calculi fragmentation). The irrigation flow or a suction flow may also assist in removal of fluid and unwanted matters (e.g., tissue debris or stone fragments), and keep the pressure of the surgical site under control, such as to maintain the pressure at substantially at a user-specified pressure level (e.g., the user-specified pressure with a tolerance such as ±5-10%). When the monitored surgical site temperature falls within a specific temperature range (e.g., below an upper “safe-operating” temperature limit), and satisfies the temperature adjustment criterion, the controller circuit 318 can control the irrigation and/or suction system 340 to automatically increase the irrigation flow into the surgical site to increase convective heat transfer, and/or increase the suction flow (or suction pressure) to withdraw the fluid away from the surgical site to improve heat dissipation and reduce the surgical site temperature. In some examples, the irrigation flow or the suction flow can be selectively activated or adjusted based on the surgical site pressure monitored via the pressure sensor 224, as described above with reference to FIG. 2.

In another example, the controller circuit 318 can generate a control signal to the irrigant treatment unit 342 to automatically adjust the temperature of the irrigant before being applied to the surgical site. The irrigant treatment unit 342 can include a cooling system (e.g., a radiator, or an in-line chiller) to cool the irrigant, or a fluid mixer to mix at least two irrigant of different temperatures. When the monitored surgical site temperature falls within a specific temperature range (e.g., below an upper “safe-operating” temperature limit), and satisfies the temperature adjustment criterion, the controller circuit 318 can control the irrigation and/or suction system 340 to automatically cool the irrigant via the cooling system or the fluid mixer. The irrigant/suction system 340 can then apply the cooled irrigate to the surgical site via the irrigation and/or suction channel 344 to improve convective heat transfer therein and effectively and efficiently reduce the surgical site temperature.

The controller circuit 318 can generate, or receive from a user, a temperature management plan that defines a prioritized order of two or more temperature control means as described above, including, for example, changing a laser output setting or one or more laser irradiation parameters, adjusting the position or orientation of the distal portion of the optical pathway (e.g., a laser fiber), activating or adjusting an irrigation flow into the surgical site and/or a suction flow away from the surgical site, or altering the temperature of the irrigant before being applied to the surgical site, among other means.

In some examples, the lithotripsy system 300 can include a camera or imaging device 325 to collect imaging signal reflected from the target in response to electromagnetic radiation (e.g., illumination light 370) of the target at or near the surgical site. The imaging signal may be transmitted to the feedback analyzer 312 through the optical pathway 360. Alternatively, the imaging signal reflected from the target or the surgical site may be transmitted through the optical pathway 334. An optical splitter may direct the reflected imaging signal to the feedback analyzer 312. The feedback analyzer 312 may include a spectrometer that may generate one or more spectroscopic properties from the imaging data. The feedback analyzer 312 may recognize the target as a calculi target or anatomical target at or near the surgical site, or classify the target as one type of tissue or one type of calculi of distinct composition using the one or more spectroscopic properties. In some examples, the feedback analyzer 312 may calculate or estimate the fiber-target distance using the spectroscopic properties. The controller circuit 318 may generate a control signal to the laser source 332 to adjust a laser output setting, a control signal to the actuator 338 to adjust the position or orientation of the distal end 346 of an irrigation and/or suction channel 344 (e.g., the fiber-tissue distance, or an aiming angle), or a control signal to the irrigation and/or suction system 340 to adjust irrigation flow or suction flow, based on the structure, composition, or type of the target.

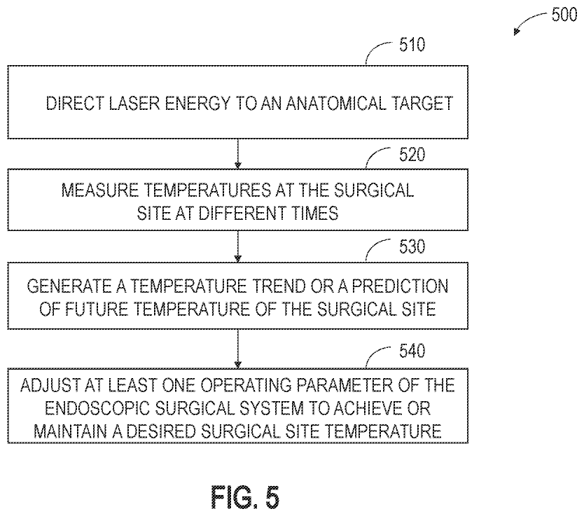

FIG. 5 is a flowchart illustrating a method 500 for controlling surgical site condition (such as surgical site temperature) during an endoscopic procedure for treating an anatomical target (e.g., soft tissue, hard tissue, cancerous tissue, or a calculi structure such as kidney or pancreobiliary or gallbladder stone). The method 500 may be implemented in and executed by the endoscopic surgical system 200, or the endoscopic laser lithotripsy system 300. Although the processes of the method 500 are drawn in one flowchart, they are not required to be performed in a particular order. In various examples, some of the processes may be performed in a different order than that illustrated herein.

At 510, laser energy (e.g., laser beams or a sequence of laser pulses) are delivered to an anatomical target. The laser energy may be generated by a laser source (such as the first laser source 106, the second laser source 116, or the laser source 332), and transmitted through an optical pathway (such as the first optical pathway 108, the second optical pathway 118, or the optical pathway 334). At 520, temperatures at the surgical site maybe sensed at different times during the procedure, such as using the temperature sensor 222. At 530, the surgical site temperature measurements may be trended over time to generate a temperature trend, such as using the temperature trending circuit 214. Additionally or alternatively, a temperature change rate can be determined using the surgical site temperature measurements. The temperature change rate (ΔT/Δt) indicates an amount of change in temperature (ΔT) over a unit time period (Δt), such as a temperature rising rate at or near the surgical site. In some examples, a prediction of future surgical site temperature at a specified future time (e.g., in 5 seconds from a present temperature measurement) can be generated based on the surgical site temperature measurements at different times in the past prior to the present measurement such as using the temperature predictor circuit 216. The prediction can be made under the assumption that the laser energy applied to the surgical site and the heat dissipation mechanisms (e.g., natural, or artificially applied such as via irrigation flow or other means of surgical site temperature control) remain unchanged. In some examples, a prediction model can be generated based on the past temperature measurements at different times. Various models may be used, including, for example, curve and surface fitting, time series regression, or machine learning (ML) approaches. The prediction model can be trained using a training dataset including surgical site temperatures measured at different times and a model type. The training process includes algorithmically adjusting model parameters (e.g., weights assigned to nodes of an input layer, output layer, or any hidden layers of a neural network model) until a convergence criteria or a training stopping criterion is satisfied. The trained predication model can be used to generate the prediction of future surgical site temperature at a specified future time. In some examples, when the measured temperature is below a pre-determined upper “safe-operating” temperature limit (max temperature), a safe-operation time window can be estimated at 530. The saft-operation time window represents time taken for the temperature to reach or exceed the “safe-operating” temperature limit.

At 540, at least one operating parameter associated with the endoscopic surgical system may be adjusted based at least in part on the generated temperature trend or the prediction of future temperature at the surgical site, such as using the controller circuit 218 or the controller circuit 318. By adjusting the at least one operating parameter, a desired temperature at the surgical site can be reached or maintained during the procedure and potential tissue thermal damage due to laser-induced overheating at the surgical site can be avoided. The adjustment of the at least one operating parameter can be carried out automatically by, for example, the controller circuit 218 or the controller circuit 318 electrically coupled to various devices of the endoscopic surgical system. Alternatively, the information about the temperature trend, the prediction of future temperatures, or the safe-operation time window may be presented to user, such as via the user interface device 250. The user may be alerted about temperature rise at the surgical site, and recommended to take proper preventive actions such as adjusting laser output or other system parameters well before the temperature reaches the “safe-operating” temperature limit.

Various temperature control means may be attempted, including, for example, changing a laser output setting or one or more laser irradiation parameters, adjusting the position or orientation of the distal portion of the optical pathway (e.g., a laser fiber), activating or adjusting an irrigation flow into the surgical site and/or a suction flow away from the surgical site, or altering the temperature of the irrigant before being applied to the surgical site, among other means, as described above with reference to FIGS. 2 and 3. In some examples, surgical site temperature can be controlled in accordance with a temperature management plan. FIG. 6A is a flowchart illustrating an example method of generating such a temperature management plan based on surgical site conditions including, for example, surgical site temperature and surgical site pressure. The temperature management plan can include a prioritized order of two or more temperature control means as described above. At 601, the temperature trend or the prediction of future surgical site temperature, such as obtained from step 530 of the method 500, can be compared against a temperature adjustment criterion. The temperature adjustment criterion can include, for example, (i) the measured surgical site temperature is within the specific temperature range (e.g., below the “safe-operating” temperature limit), and (ii) at least one of: the temperature rising rate exceeds a rate threshold, the prediction of future temperate in next X seconds would exceed the “safe-operating” temperature limit; or the estimated safe-operation time window is shorter than a pre-determined threshold window length. At 602, pressure can be sensed at the surgical site, such as using the pressure sensor 224. At 603, the sensed surgical site pressure, P, can be compared to a pre-determine or user-specified upper pressure limit Pₘₐₓ, also referred to as a maximum allowable pressure. At 604, a temperature management plan can be determined based on the temperature check at 601 and the pressure check at 603. Availability (e.g., irrigant cooling system) and efficiency of temperature control means, or potential adverse effects on the surgical site, may also be considered to determine an individualized temperature management plan for the patient. The temperature management plan can include a prioritized order of two or more temperature control means as described above, including, for example, changing a laser output setting or one or more laser irradiation parameters, adjusting the position or orientation of the distal portion of the optical pathway (e.g., a laser fiber), activating or adjusting an irrigation flow into the surgical site and/or a suction flow away from the surgical site, or altering the temperature of the irrigant before being applied to the surgical site, among other means. In an example as shown in FIG. 6A, when the temperature trend or the prediction of future surgical site temperature satisfies the temperature adjustment criterion at 601, the prioritized order between adjusting the laser output setting and adjusting irrigation or suction flow can be based at least in part on whether the surgical site pressure, P, reaches a level substantially close to the upper pressure limit Pₘₐₓ at 605. If the surgical site pressure P is substantially below Pₘₐₓ (e.g., the difference between P and Pₘₐₓ exceeds a threshold), the irrigation flow rate can be increased at 606. If the surgical site pressure P is substantially close to Pₘₐₓ (e.g., within a user-specified or pre-determined margin of Pₘₐₓ, e.g., ±10%), then the priority of temperature control means can be determined based on whether an option to increase suction flow is available at 607. If the suction flow is not available or not activated by the user, then the laser output setting can be adjusted, such as by decreasing laser output at 608. However, if at 607 the suction flow is available and activated by the user, then the suction flow rate can be increased at 609. Following the temperature control operations at any of steps 606, 608, and 609, other temperature control means may be attempted in an on-demand mode (e.g., activated by the user). The monitoring of surgical site temperature can be continued at 520.

FIG. 6B is a flowchart illustrating an example of the temperature management plan with prioritized means for controlling surgical site temperature, which can be an embodiment of steps 540 of adjusting at least one operating parameter associated with the endoscopic surgical system to achieve or maintain substantially a desired temperature at the surgical site. The temperature trend, the prediction of a future surgical site temperature, or the estimated safe-operation time window, generated at 530, can be used to determine whether a temperature adjustment criterion is satisfied at 610. Such temperature adjustment criterion can include, for example, (i) the measured surgical site temperature is within the specific temperature range (e.g., below the “safe-operating” temperature limit), and (ii) at least one of: the temperature rising rate exceeds a rate threshold, the prediction of future temperate in next X seconds would exceed the “safe-operating” temperature limit; or the estimated safe-operation time window is shorter than a pre-determined threshold window length. If the criterion is not satisfied at 610, then the surgical site temperature is deemed normal and no parameter is to be adjusted, and the monitoring of surgical site temperature can be continued at 620. If the criterion is satisfied at 610, then an option for cooling the irrigant before flowing into the surgical site is provided at 620. If irrigant cooling option is available and selected (e.g., by the user), then at 622 irrigant can be cooled before reaching the surgical site, such as using a cooling system (e.g., a radiator or an in-line chiller included in the irrigant treatment unit 342), or by mixing at least two irrigant sources of different temperatures. The cooled irrigate can be applied to the surgical site to improve convective heat transfer therein. The monitoring of surgical site temperature can be continued at 520.

If the irrigant cooling option is not available or not selected at 620, then an option for using an irrigation and/or suction system (such as the irrigation and/or suction system 240 or the irrigation and/or suction system 340) is provided at 630. As discussed above with reference to FIGS. 2 and 3, the irrigation and/or suction system can provide an irrigation inflow to the surgical site, and/or a suction flow (outflow) of fluid out of the surgical site. In addition to assisting in removal of the tissue debris, stone fragments, and other unwanted matters during the procedure, the irrigation flow and suction flow also have a cooling effect on the tissue at or near the surgical site. If the irrigation and/or suction option is not available or not selected (e.g., by the user) at 630, then laser output settings can be adjusted at 632. For example, an average power of laser pulses can be reduced, such as by reducing one or more of a pulse width of a laser pulse, a peak power of a laser pulse, or a pulse frequency representing a number of laser pulses per unit time. Reducing the average power of laser pulses can decrease the laser-induced heating effect at or near the surgical site, thereby preventing tissue thermal damage and improving patient safety during the procedure. In some examples, a position or orientation of a distal portion of an optical pathway relative to the anatomical target at the surgical site can be adjusted, such as via the actuator 338 to adjust the position or orientation of the distal end 336 of the optical pathway 334. The position or orientation can be adjusted to increase the fiber-target distance and/or to increase the aiming angle, thereby reducing the density of the laser energy incident on the surgical site and the laser-induced heat transferred into the surgical site. The monitoring of surgical site temperature can be continued at 520.

If the irrigation and/or suction option is available and selected at 630, then at 640 a pressure can be sensed at the surgical site, such as using the pressure sensor 224. Depending on the sensed pressure (P) at the surgical site, one or both of the irrigation flow or the suction flow may be selectively activated or adjusted to achieve temperature control at the surgical site temperature. At 650, the measured surgical site pressure is compared to a pre-determine or user-specified upper pressure limit Pₘₐₓ. If the measured surgical site pressure, P, exceeds the Pₘₐₓ (P>Pₘₐₓ), then at 652 only the suction flow rate (but not the irrigation flow rate) is increased to lower the surgical site temperature. Additionally or alternatively, the irrigation flow rate may be reduced to reduce the pressure at the surgical site. As an increase in irrigation flow into the surgical site may introduce a positive pressure change at or near the surgical site, further increase in irrigation flow should be avoided to prevent further increase in surgical site pressure. If the measured surgical site pressure is lower than Pₘₐₓ, the measured surgical site pressure can be further compared to a pre-determine or user-specified a lower pressure limit Pₘᵢₙ at 660. If the measured surgical site pressure is within a range defined by Pₘᵢₙ and Pₘₐₓ (Pₘᵢₙ<P<Pₘₐₓ), then at 670 one or both of the irrigation flow rate or the suction flow rate can be increased to lower the surgical site temperature. However, if at 660 the measured surgical site pressure falls below the lower pressure limit Pₘᵢₙ (P<Pₘᵢₙ), then at 662 only the irrigation flow rate into the surgical site (but not the suction flow) is increased to lower the surgical site temperature, but avoid increasing the suction flow rate to prevent further decrease in surgical site pressure. Additionally or alternatively, the suction flow may be reduced to increase the pressure at the surgical site. As an increase in suction flow may introduce a negative pressure change at or near the surgical site, further increase in suction flow should be avoided to prevent further decrease in surgical site pressure. After the adjustment of irrigation or suction flow at 652, 662, or 670, the monitoring of surgical site temperature can be continued at 620.

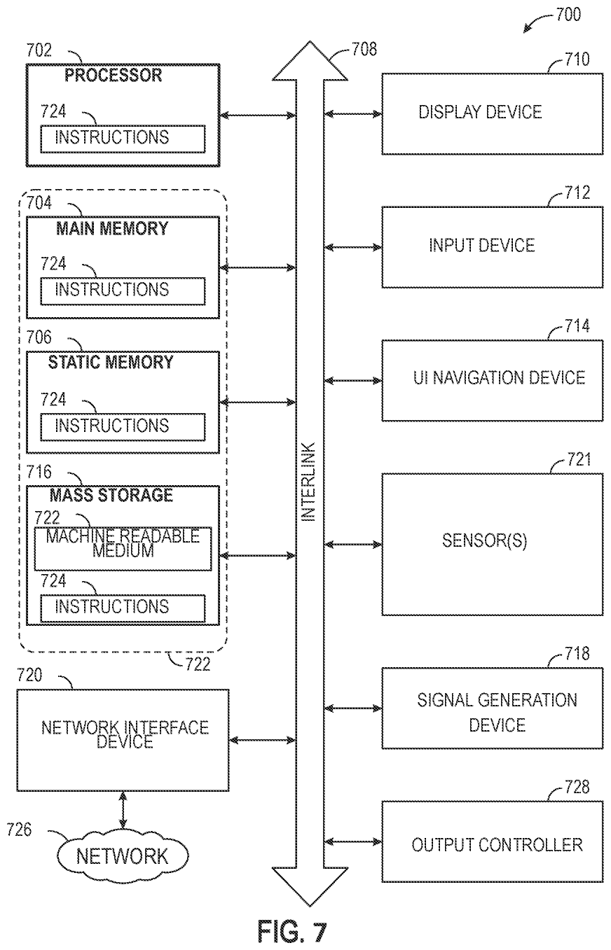

FIG. 7 illustrates generally a block diagram of an example machine 700 upon which any one or more of the techniques (e.g., methodologies) discussed herein may perform. Portions of this description may apply to the computing framework of various portions of the endoscopic surgical system 200 or the endoscopic surgical system 300.

In alternative embodiments, the machine 700 may operate as a standalone device or may be connected (e.g., networked) to other machines. In a networked deployment, the machine 700 may operate in the capacity of a server machine, a client machine, or both in server-client network environments. In an example, the machine 700 may act as a peer machine in peer-to-peer (P2P) (or other distributed) network environment. The machine 700 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 700 may include a hardware processor 702 (e.g., a central processing unit (CPU), a graphics processing unit (GPU), a hardware processor core, or any combination thereof), a main memory 704 and a static memory 706, some or all of which may communicate with each other via an interlink (e.g., bus) 708. The machine 700 may further include a display unit 710 (e.g., a raster display, vector display, holographic display, etc.), an alphanumeric input device 712 (e.g., a keyboard), and a user interface (UI) navigation device 714 (e.g., a mouse). In an example, the display unit 710, input device 712 and UI navigation device 714 may be a touch screen display. The machine 700 may additionally include a storage device (e.g., drive unit) 716, a signal generation device 718 (e.g., a speaker), a network interface device 720, and one or more sensors 721, such as a global positioning system (GPS) sensor, compass, accelerometer, or other sensors. The machine 700 may include an output controller 728, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 716 may include a machine readable medium 722 on which is stored one or more sets of data structures or instructions 724 (e.g., software) embodying or utilized by any one or more of the techniques or functions described herein. The instructions 724 may also reside, completely or at least partially, within the main memory 704, within static memory 706, or within the hardware processor 702 during execution thereof by the machine 700. In an example, one or any combination of the hardware processor 702, the main memory 704, the static memory 706, or the storage device 716 may constitute machine readable media.

While the machine-readable medium 722 is illustrated as a single medium, the term “machine readable medium” may include a single medium or multiple media (e.g., a centralized or distributed database, and/or associated caches and servers) configured to store the one or more instructions 724.

The term “machine readable medium” may include any medium that is capable of storing, encoding, or carrying instructions for execution by the machine 700 and that cause the machine 700 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EPSOM)) and flash memory devices; magnetic disks, such as internal hard disks and removable disks; magneto-optical disks; and CD-ROM and DVD-ROM disks.

The instructions 724 may further be transmitted or received over a communication network 726 using a transmission medium via the network interface device 720 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 720 may include one or more physical jacks (e.g., Ethernet, coaxial, or phone jacks) or one or more antennas to connect to the communication network 726. In an example, the network interface device 720 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 700, and includes digital or analog communications signals or other intangible medium to facilitate communication of such software.

### Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. An endoscopic surgical system, comprising:
an endoscopic surgical device controllably coupled to a medical instrument for delivering energy to an anatomical target at a surgical site during a procedure;
a temperature sensor for measuring temperatures in a vicinity of the surgical site at different times during the procedure; and
a controller circuit configured to:
generate a temperature trend or a prediction of future temperature at the surgical site based at least in part on the temperature measurements at the different times; and
based at least in part on the generated temperature trend or the prediction of future temperature at the surgical site, adjust at least one operating parameter associated with the endoscopic surgical system to achieve or maintain substantially a desired temperature at the surgical site during the procedure.

2. The endoscopic surgical system of claim 1, wherein the medical instrument comprises at least one laser system for delivering laser energy to a calculi target at the surgical site when the endoscopic surgical system operates in accordance with the adjusted at least one operating parameter.

3. The endoscopic surgical system of claim 1, wherein the controller circuit is further configured to:
determine a temperature change rate at the surgical site using the generated temperature trend; and
adjust the at least one operating parameter in response to the determined temperature change rate exceeding a predetermined threshold.

4. The endoscopic surgical system of claim 1, wherein the controller circuit is further configured to:
generate a trained prediction model using the temperature measurements at the different times;
generate the prediction of future temperature at the surgical site further using the trained prediction model; and
adjust the at least one operating parameter in response to the prediction of future temperature exceeding a temperature threshold.

5. The endoscopic surgical system of claim 1, wherein the controller circuit is further configured to:
estimate a safe-operation time window using the temperature measurements at the different times, the safe-operation time window representing an estimate of time taken to reach a safe-operating temperature limit at the surgical site; and
adjust the at least one operating parameter in response to the estimated safe-operation time window falling below a time threshold.

6. The endoscopic surgical system of claim 2, wherein the at least one operating parameter to be adjusted includes a laser output setting of the at least one laser system,
wherein the controller circuit is further configured to toggle between at least first and second pre-determined pulse profiles based at least in part on the generated temperature trend or the prediction of future temperature at the surgical site, the second pre-determined pulse profile having a lower average power than the first pre-determined pulse profile.

7. The endoscopic surgical system of claim 6, wherein to adjust the laser output setting, the controller circuit is further configured to reduce an average power of laser pulses delivered to the surgical site in response to (i) the generated temperature trend indicating an increase in temperature at a rate exceeding a rate threshold, or (ii) the prediction of future temperature exceeding a temperature threshold.

8. The endoscopic surgical system of claim 7, wherein to reduce the average power of laser pulses includes to reduce at least one of:
a pulse width of a laser pulse;
a peak power of a laser pulse; and
a pulse frequency representing a number of laser pulses per unit time.

9. The endoscopic surgical system of claim 1, comprising an irrigation and/or suction system configured to provide irrigant into, and suction of fluid from, the surgical site,
wherein to adjust the at least one operating parameter, the controller circuit is further configured to adjust, via the irrigation and/or suction system, at least one of an irrigation flow or a suction flow based at least in part on the generated temperature trend or the prediction of future temperature at the surgical site.

10. The endoscopic surgical system of claim 9, wherein the controller circuit is configured to increase at least one of the irrigation flow or the suction flow in response to (i) the generated temperature trend indicating an increase in temperature at a rate exceeding a rate threshold, or (ii) the prediction of future temperature exceeding a temperature threshold.

11. The endoscopic surgical system of claim 9, further comprising a pressure sensor configured to sense a pressure at the surgical site during the procedure,
wherein the controller circuit is further configured to selectively increase the irrigation flow or the suction flow via the irrigation and/or suction system, including to:
increase the suction flow or decrease the irrigation flow when the sensed pressure exceeds an upper pressure limit;
increase one or both of the irrigation flow or the suction flow when the sensed pressure is within a range defined by the upper pressure limit and a lower pressure limit; and
increase the irrigation flow or decrease the suction flow when the sensed pressure falls below the lower pressure limit.

12. The endoscopic surgical system of claim 9, comprising an irrigant treatment unit configured to alter a temperature of the irrigant,
wherein the controller circuit is further configured to generate a control signal to the irrigant treatment unit to adjust a temperature of the irrigant before reaching the surgical site based at least in part on the generated temperature trend or the prediction of future temperature at the surgical site.

13. The endoscopic surgical system of claim 12, wherein the irrigant treatment unit includes a cooling system configured to, under the control of the controller circuit, cool the irrigant before reaching the surgical site in response to (i) the generated temperature trend indicating an increase in temperature at a rate exceeding a rate threshold, or (ii) the predicted future temperature exceeding a temperature threshold.

14. The endoscopic surgical system of claim 12, wherein the irrigant treatment unit includes a fluid mixer configured to, under the control of the controller circuit, mix at least two irrigant sources of different temperatures before reaching the surgical site in response to (i) the generated temperature trend indicating an increase in temperature at a rate exceeding a rate threshold, or (ii) the predicted future temperature exceeding a temperature threshold.

15. The endoscopic surgical system of claim 2, wherein the endoscopic surgical device includes an optical pathway with an adjustable distal portion, the optical pathway configured to direct the laser energy to the anatomical target,
wherein the controller circuit is further configured to, based at least in part on the generated temperature trend or the prediction of future temperature at the surgical site, generate a control signal to an actuator coupled to the optical pathway to adjust a position or orientation of the distal portion of the optical pathway relative to the anatomical target.

16. The endoscopic surgical system of claim 1, wherein the at least one operating parameter associated with the endoscopic surgical system comprises at least one of:
a temperature of an irrigant before being applied to the surgical site;
an irrigation flow rate;
a suction flow rate; or
a laser output setting of a laser system.

17. The endoscopic surgical system of claim 16, wherein the controller circuit is further configured to perform the adjustment with a bias toward one of the operating parameters based at least in part on at least one of the generated temperature trend, the prediction of future temperature, or a pressure at the surgical site.

18. The endoscopic surgical system of claim 17, wherein the controller circuit is further configured to:
upon determining that the pressure at the surgical site is substantially below a maximal allowable pressure, adjust at least one of the irrigation flow rate or the suction flow rate prior to adjusting the laser output setting; and
upon determining that the pressure at the surgical site is substantially close to a maximal allowable pressure, adjust the laser output setting prior to adjusting the irrigation flow rate or the suction flow rate.

19. A method for controlling temperature at a surgical site of a patient during an endoscopic procedure using an endoscopic surgical system, the method comprising:
directing energy produced by a medical instrument to an anatomical target at the surgical site;
measuring temperatures in a vicinity of the surgical site at different times during the procedure;
generating a temperature trend or a prediction of future temperature at the surgical site based at least in part on temperature measurements at the different times; and
based at least in part on the generated temperature trend or the prediction of future temperature at the surgical site, adjusting at least one operating parameter associated with the endoscopic surgical system to achieve or maintain substantially a desired temperature at the surgical site during the procedure.

20. The method of claim 19, comprising determining a temperature change rate at the surgical site using the generated temperature trend,
wherein adjusting the at least one operating parameter is in response to the determined temperature change rate exceeding a predetermined threshold of a temperature change rate.

21. The method of claim 19, comprising generating a trained prediction model using the temperature measurements at the different times,
wherein generating the prediction of future temperature at the surgical site is by using the trained prediction model,
wherein adjusting the at least one operating parameter is in response to the prediction of future temperature exceeding a predetermined temperature threshold.

22. The method of claim 19, comprising estimating a safe-operation time window using the temperature measurements at the different times, the safe-operation time window representing an estimate of time taken to reach a safe-operating temperature limit at the surgical site,
wherein adjusting the operating parameter is in response to the estimated safe-operation time window falling below a time threshold.

23. The method of claim 19, wherein adjusting the at least one operating parameter includes, via at least one laser system, reducing an average power of laser pulses delivered to the surgical site in response to (i) the generated temperature trend indicating an increase in temperature at a rate exceeding a rate threshold, or (ii) the prediction of future temperature exceeding a temperature threshold.

24. The method of claim 19, wherein adjusting the at least one operating parameter includes, via an irrigation and/or suction system, increasing at least one of an irrigation flow of irrigant into the surgical site or a suction flow of fluid out of the surgical site, in response to (i) the generated temperature trend indicating an increase in temperature at a rate exceeding a rate threshold, or (ii) the prediction of future temperature exceeding a temperature threshold.

25. The method of claim 24, further comprising sensing a pressure at the surgical site during the procedure using a pressure sensor,
wherein adjusting at least one of the irrigation flow or the suction flow includes:
increasing the suction flow or decrease the irrigation flow when the sensed pressure exceeds an upper pressure limit;
increasing one or both of the irrigation flow or the suction flow when the sensed pressure is within a range defined by the upper pressure limit and a lower pressure limit; and
increasing the irrigation flow or decrease the suction flow when the sensed pressure falls below the lower pressure limit.

26. The method of claim 19, wherein adjusting the at least one operating parameter includes adjusting, via an irrigant treatment unit coupled to an irrigation and/or suction system, a temperature of an irrigant before flowing into the surgical site based at least in part on the generated temperature trend or the prediction of future temperature at the surgical site.

27. The method of claim 19, wherein adjusting the at least one operating parameter includes adjusting a position or orientation of a distal portion of an optical pathway relative to the anatomical target at the surgical site, and directing the energy to the anatomical target via the optical pathway.
